SEQUENCE LISTING (2 sequences)

SEQ ID NO: 1 - DNA, 30 bp, Artificial Sequence
  DNA probe.
caagcttatcgataccgtcgacctcgaggg

SEQ ID NO: 2 - DNA, 30 bp, Artificial Sequence
  DNA complementary with DNA probe defined by SEQ.  No. 1.
ccctcgaggtcgacggtatcgataagcttg

(19) United States
(12) Patent Application Publication
(10) Pub. No.: US 2003/0162283 A1
(43) Pub. Date: Aug. 28, 2003

(54) Circulating type biochemical reaction apparatus

(75) Inventors: Norihito Kuno, Tsurugashima (JP); Kenko Uchida, Tokyo (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/206,206

(22) Filed: Jul. 29, 2002

(30) Foreign Application Priority Data
Feb. 22, 2002 (JP) P2002-045573

(51) Int. Cl. C12M 1/34

(52) U.S. Cl. 435/287.2; 435/288.3; 435/293.1

(57) ABSTRACT

The invention provides a circulating type biochemical reaction apparatus for carrying out hybridization efficiently and uniformly having a plate-like member 5 having a channel 6 for circulating a sample solution, a flow-in port 7, a flow-out port 8, flow-rectifying protrusions 9 formed thereon, and a plate-like member having a hollow 3 formed thereon for holding a probe substrate 1 to be combined and fixed together to form a unit. The unit is disposed with an inclination from a horizontal plane, with the flow-in port 7 coming at a lower level than the flow-out port 8. The sample solution is fed through the flow-in port 7 to be poured into the channel and circulated. The circulation improves the reaction efficiency and thereby increases signal intensities and reduces reaction time. Furthermore, the reaction proceeds uniformly such that the signal intensity is also.

### FIELD OF THE INVENTION

This invention relates to a circulating type biochemical reaction apparatus having a channel for circulating a sample solution containing biomolecules capable of interacting with a probe immobilized on a substrate.

### BACKGROUND OF THE INVENTION

As a result of the progress of the human genome sequencing project and the finishing of a first draft sequence, one of the subjects of inquiry attracting attention in the post-genome-sequence era is to analyze the changes in gene expression and in protein expression. Thus, the microarray technique for analyzing the time of expression of a gene and in situ hybridization technique for analyzing the site or tissue of expression of a gene become increasingly important. According to the microarray or in situ hybridization technique, a probe or a sample of a nucleic acid, protein, tissue section or like is immobilized or trapped on a substrate or a base plate, then the sample-probe hybridization occurs, and whether there is a change in the level of a nucleic acid or protein in the sample is analyzed.

Generally, these hybridization reactions are carried out by dropping a hybridization solution containing a probe or a sample onto a substrate with a sample or a probe immobilized thereon, covering the substrate with a cover glass such that the hybridization solution may not evaporate, placing the substrate in a wet box or a tightly closed cassette, and maintaining the substrate at a constant temperature for a fairly long period of time (not shorter than 12 hours).

Reaction apparatus have so far been developed for carrying out the above hybridization reactions with ease. Closed cassettes for carrying out the hybridization reactions while maintaining a DNA microarray set with a hybridization solution by placing a cover glass have been known.

However, in placing a cover glass, for standing still, on the hybridization solution to cover the solution therewith, the contact of the cover glass with the DNA spot on the substrate causes partial or extensive depletion of the DNA spot. This is one of the factors affecting the spot intensity after hybridization thereby decreasing the reliability of the data obtained.

Therefore, in lieu of a cover glass, for standing still, on the hybridization solution, a special cover glass has been known to provide a space with a height of 0.02 mm therein for retaining the hybridization solution on the spotted surface of the DNA microarray.

However, in cases where the above-mentioned cassette or cover glass is used, no substantial movement of the hybridization solution is allowed to occur on the substrate. The frequency of collision between the probe immobilized on the substrate and the sample in the solution is kept low, as such, the hybridization efficiency cannot be improved. Therefore, a fairly long time (at least 12 hours) is required for the hybridization reaction. Further, the data reliability problem due to inhomogeneous hybridization, especially low reproducibility, is one of the problems encountered in carrying out the hybridization with a probe immobilized on a substrate.

The hybridization technique is using so far been used in molecular biological studies a membrane as a sample immobilizing support. It is well known that shaking and/or stirring of the hybridization solution is effective in reducing the hybridization time and/or securing uniformity in hybridization signals. Therefore, hybridization ovens which can shake or stir solutions by a rotisserie type rotating device or a shaking platform are currently used in carrying out the hybridization with membranes.

For the above-mentioned technique of hybridization with biomolecules immobilized on a substrate as well, hybridization apparatus for shaking or stirring a hybridization solution have been developed in recent years to reduce reaction time and/or attaining uniform hybridization. For example, an apparatus, for in situ hybridization reaction is applicable to tissue sections (U.S. Pat. No. 5,650,327). This apparatus has an automated reagent distributing function and, in addition, a unique liquid cover slip, and an air mixer, by which the hybridization solution is stirred on a slide glass with a tissue section immobilized thereon, so as to realize that the hybridization reaction in a highly efficient manner.

As a hybridization apparatus for a DNA microarray, an apparatus is described in U.S. Pat. No. 6,238,910. In this apparatus, the hybridization solution in the reaction vessel is agitated with air (for causing reciprocal liquid shaking) to improve the reactivity in hybridization. Another apparatus for effecting similar liquid shaking is also available.

Those prior art hybridization apparatus for shaking and/or stirring a hybridization solution shaking and/or stirring function are effective in improving the hybridization efficiency as compared with the apparatus without shaking or stirring. However, as regards the above-mentioned U.S. Pat. No. 5,650,327, the stirring of the reaction mixture by means of an air jet is indeed sufficient in the middle portion of the slide but may be insufficient in the peripheral portion of the slide glass. Therefore, when a sample or a probe is immobilized on the whole surface of the slide glass, in particular in the case of a DNA microarray, the hybridization in the peripheral portion of the slide glass may possibly become inhomogeneous.

In cases where the hybridization solution is shaken in a reciprocating manner (back and forth alternately), as in the above-mentioned U.S. Pat. No. 6,238,910 or apparatus for effecting liquid shaking, once a bubble or bubbles enter or are formed in the hybridization solution, the hybridization reaction proceeds with bubbles (always contained in the solution). Such bubbles contained in the solution become one of the main factors causing inhomogeneous hybridization For attaining homogeneous hybridization, it is required that bubbles in the hybridization solution be trapped and removed therefrom.

Further, in the prior art apparatus, a plurality of reaction vessels are controlled by one single main controller equipped with agitation pumps. The number of reaction vessels is invariable and can not be changed to an arbitrary number. In actual test experiments, however, the number of samples to be tested often varies from experiment to experiment, hence it is not always equal to the number of available reaction vessels. Therefore, it is difficult to flexibly change according to the number of samples to be tested.

Accordingly, it is an object of the present invention to provide a circulating type biochemical reaction apparatus in which the hybridization reaction with biomolecules immobilized on a substrate can be carried out efficiently and uniformly.

Another object of the invention is to provide a circulating type biochemical reaction apparatus capable of flexibly coping with the change in the number of samples.

### SUMMARY OF THE INVENTION

A circulating type biochemical reaction apparatus is provided with a substrate (probe substrate) with a plurality of mutually separated sections each having at least one probe immobilized thereon for selective binding to a target substance in a sample. The substrate is held by a first plate-like member. A second plate-like member has a flow-in port for allowing a sample solution containing a sample and reagents, which are necessary for enabling target substance-probe binding, to flow into the apparatus as well as a flow-out port for allowing the sample solution to flow out. A canal or channel for circulating the sample solution is formed between the immobilized probe-bearing surface of the substrate held by the first plate-like member and the second plate-like member. A pump for circulating the sample solution is established with the first plate-like member and/or the second plate-like member. The flow-in port is disposed lower than the flow-out port. The pump circulates the sample solution by causing the sample solution to flow into the channel from below and flow out from the flow-out port. While the sample solution is circulated, the reaction is allowed to proceed by which the target substance is bound to the probe. Additionally, the first plate-like member and the second plate-like member are disposed with an inclination from a horizontal plane such that the bubbles enter into or are formed in the sample solution in the channel move upward. Moreover, to cope with the bubbles enter into or are formed in the sample solution in the channel, the disposition of a site for bubble trapping is preferable.

The above constitution makes it possible to introduce the sample solution into the channel and circulate the same without allowing bubbles to mix therein. The circulation improves the reaction efficiency thereby increasing, the signal intensity, reducing the reaction time to proceed uniformly such that the quantitativity of the signal intensity is improved. Thus, the target substance-probe binding reaction, for example, the hybridization reaction, can be carried out efficiently and uniformly.

When the target substance is (1a) a single-stranded or double-stranded nucleic acid, (2a) an antibody, (3a) an antigen, (4a) a receptor, (5a) a ligand, (6a) an enzyme, (7a) a substrate or (8a) a nucleic acid, a probe substrate with (1b) a nucleic acid, (2b) an antigen, (3b) an antibody, (4b) a ligand, (5b) a receptor, (6b) a substrate, (7b) an enzyme or (8b) a peptidyl nucleic acid immobilized as a probe is used respectively.

### BRIEF DESCRIPTION OF THE DRAWINGS

FIGS. 1A to 1C show the constitution of a first embodiment (Example 1) of the circulating type biochemical reaction apparatus of the present invention.

FIGS. 2A to 2C show the constitution of the first plate-like member for holding a probe substrate, to be used in the circulating type biochemical reaction apparatus of the invention as described in Example 1.

FIGS. 3A to 3D show the constitution of the second plate-like member for holding the probe substrate, to be used in the circulating type biochemical reaction apparatus of the invention as described in Example 1.

FIGS. 4A to 4H illustrate how the liquid flows in the channel of the circulating type biochemical reaction apparatus of the invention as described in Example 1.

FIG. 5 shows some results obtained by mounting a DNA microarray on the circulating type biochemical reaction apparatus of the invention as described in Example 1 of circulating a sample solution to thereby perform hybridization.

FIG. 6 shows some results obtained in Example 1 by carrying out DNA microarray hybridization with a commercially available reaction apparatus without stirring or circulation.

FIG. 7 is a cross-sectional view showing the constitution of a second embodiment (Example 2) of the circulating type biochemical reaction apparatus of the present invention, which is equipped with a liquid feeding pump.

FIG. 8 is a cross-sectional view showing the construction of a third embodiment (Example 3) of the circulating type biochemical reaction apparatus of the invention, which is equipped with a liquid feeding pump, a washing solution reservoir and a waste liquor reservoir.

FIGS. 9A to 9B show the disposition of protrusions formed on the second plate-like member for rectifying the liquid flow in the circulating type biochemical reaction apparatus of the invention as described in Example 4.

FIGS. 10A and 10B show the disposition of a plurality of linear protrusions formed on the second plate-like member for rectifying the liquid flow in the circulating type biochemical reaction apparatus of the invention as described in Example 4.

FIGS. 11A and 11B show another example of the disposition of a plurality of linear protrusions formed on the second plate-like member for rectifying the liquid flow in the circulating type biochemical reaction apparatus of the invention as described in Example 4.

FIG. 12 shows the disposition of a plurality of flow-in ports and of flow-out ports as formed on the second plate-like member in the circulating type biochemical reaction apparatus of the invention as described in Example 5.

FIG. 13 is a plan view illustrating the disposition of sections on the probe substrate to be used in the circulating type biochemical reaction apparatus of the invention as described in the examples.

FIG. 14 illustrates the approximate positions of domains A1 to A3, B1 to B3, and C1 to C3, where a probe was spotted for fluorescence detection in Example 1 according to the invention.

FIG. 15 shows a table of the mean fluorescence intensity values and standard deviations, and relative standard derivations as obtained in the respective spot domains in Example 1 according to the invention, with (FIG. 5) or without (FIG. 6) circulation of the sample solution in the step of hybridization.

### DETAILED DESCRIPTION OF THE PREFERRED EMBODIMENTS

In the following, several embodiments of the present invention are described in detail referring to the drawings.

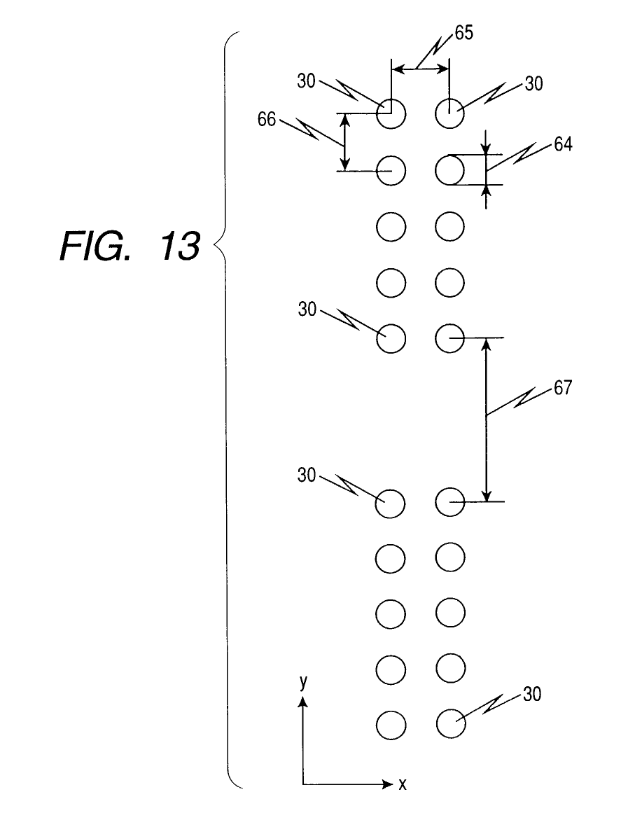

FIG. 13 is a plan view illustrating the disposition of probe immobilization sections on the probe substrate to be used in the circulating type biochemical reaction apparatus of the invention as described in the following examples. In FIG. 13, the disposition of sections 30 is partly shown, and the mutually separated sections 30 are disposed on the substrate in the x and y directions. The distance 64 indicates the diameter of each circular section 30, and the distances 65, 66, and 67 respectively show the center-to-center distances between neighboring sections 30. Each section on which a probe is immobilized has a diameter of about 100 μm to 350 μm. The probe substrate 1 is plate-like and has an area size of 22 mm×75 mm, with a thickness of 1.0 mm to 2.0 mm. A maximum of about 15,000 sections 30 for probe immobilization can be formed on the probe substrate 1. The probe substrates 1 used in the following examples are made of glass and have an area size of 22 mm×75 mm, with a thickness of 1.0 mm. The diameter 64 of each section was about 350 μm. The center-to-center distances 65 and 66 between sections 30 are each about 600 μm in the x and y directions. A distance 67 of 2 mm is kept per every 5 sections in the y direction. The following examples are described as examples, where a single-stranded nucleic acid is used as the probe and a single-stranded nucleic acid capable of binding complimentarily with the former single-stranded nucleic acid as the sample. The principle of the following examples can be applied also to those cases where a double-stranded nucleic acid having a single-stranded protruding end is the target substance.

### EXAMPLE 1

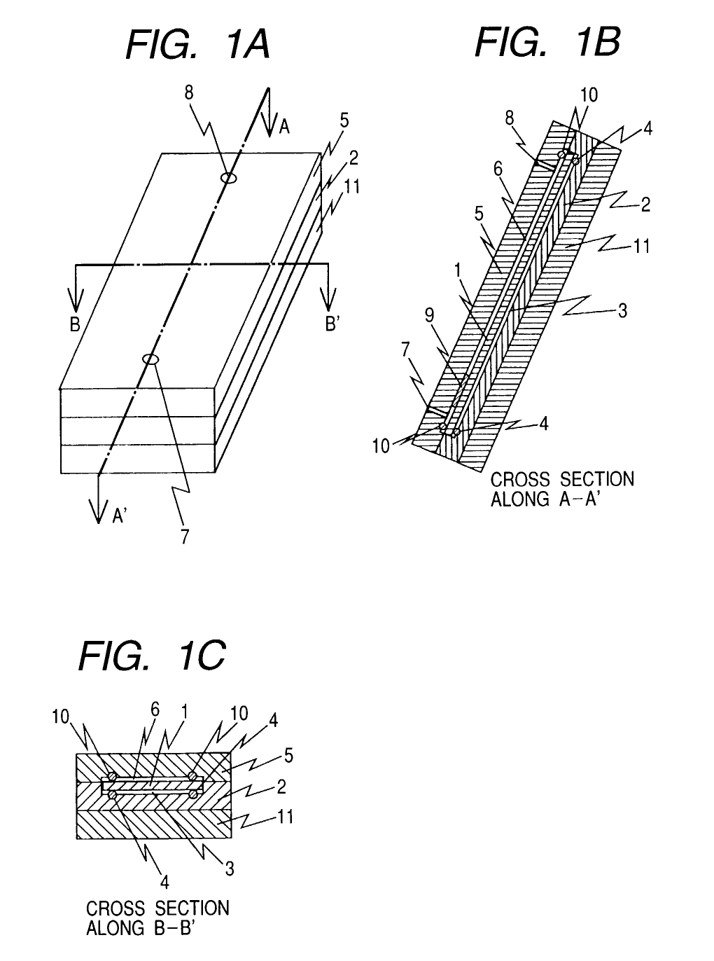

FIGS. 1A to 1C show the constitution of the circulating type biochemical reaction apparatus of the present invention as used in Example 1. FIG. 1A is a perspective view, FIG. 1B a cross-sectional view along A-A′ in FIG. 1A, and FIG. 1C a sectional view along B-B′ in FIG. 1A.

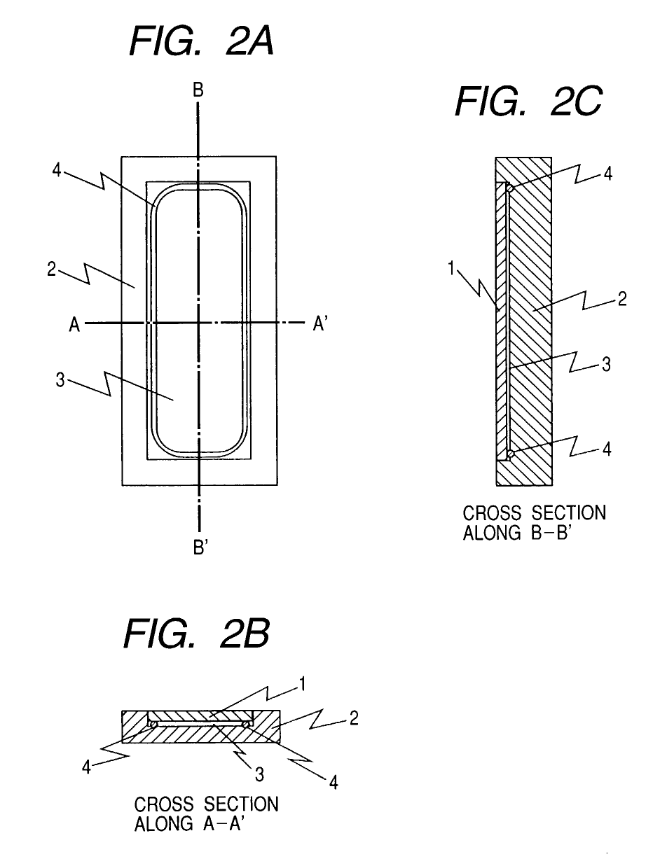

FIGS. 2A to 2C show the constitution of the first plate-like member for holding a probe substrate, which is to be used in the circulating type biochemical reaction apparatus of the invention as described in Example 1. FIG. 2A is a plan view, FIG. 2B a sectional view along A-A′ in FIG. 2A, and FIG. 2C a sectional view along B-B′ in FIG. 2A.

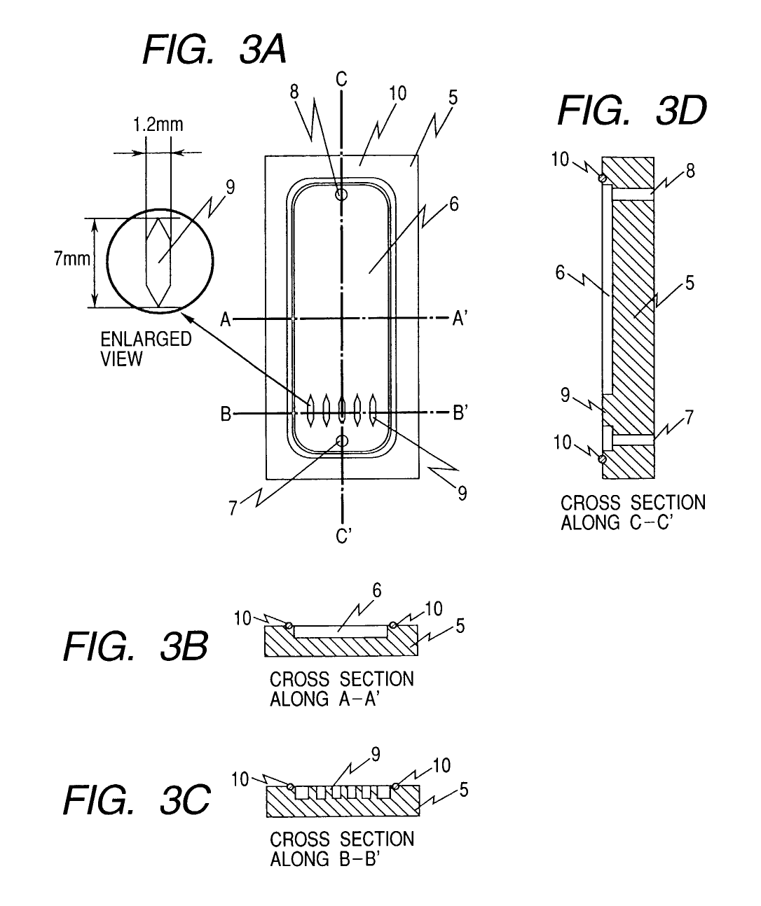

FIGS. 3A to 3D show the constitution of the second plate-like member for holding the probe substrate, which is to be used in the circulating type biochemical reaction apparatus of the invention as described in Example 1. FIG. 3A is a plan view with a partial enlarged view, FIG. 3B a sectional view along A-A′ in FIG. 3A, FIG. 3C a cross-sectional view along B-B′ in FIG. 3A, and FIG. 3D a cross-sectional view along C-C′ in FIG. 3A.

In Example 1, the circulating type biochemical reaction apparatus of the present invention is constituted of a plate-like member (first plate-like member) 2, a plate-like member (second plate-like member) 5, a probe substrate 1, a heating/cooling unit 11, and O rings 4, 10.

In the circulating type biochemical reaction apparatus of the invention (Example 1), the plate-like member 5 on which a channel 6 for sample solution circulation, a flow-in port 7, a flow-out port 8 and protrusions 9 for rectifying the liquid flow are formed, and the plate-like member 2 with a hollow 3 formed thereon for holding the probe substrate 1 are combined and fixed together to form a unit. This unit is disposed with an inclination relative to a horizontal plane (about 5° to 90°, and preferably 45° to 90° from the horizontal plane), with the flow-in port 7 being positioned at a level below the flow-out port 8. A sample solution is fed through the flow-in port 7 to be poured into the channel and circulated.

As shown in FIG. 2, the plate-like member 2 has a hollow 3 for receiving and holding the probe substrate 1 via the O ring 4 disposed in the peripheral portion of the hollow 3. The bottom of the hollow 3 has an area size of 25 mm×77 mm and the depth of the hollow 3 is 1.2 mm.

As shown in FIGS. 3A to 3D, the plate-like member 5 forms, together with the probe substrate 1, a channel 6 through which a sample solution containing a sample and reagents necessary for allowing the target substance to bind to the probe is to flow, and has a flow-in port 7 penetrating the plate-like member 5 and allowing the sample solution to flow into the channel, a flow-out port 8 penetrating the plate-like member 5 and allowing the sample solution to flow out of the channel, and hexagonal protrusions 9 for controlling the flow of the solution and causing the solution to flow uniformly through the channel. The channel 6 formed between the probe substrate 1 and the member 5 has an area size of 18 mm×68 mm and a depth of 100 μm. The flow-in port 7 and the flow-out port 8 each has an inside diameter of 1 mm. The protrusions 9 are disposed symmetrically on both sides of the centerline connecting the center of the flow-in port 7 with that of the flow-out port 8. The protrusions have a bottom length of 7 mm in parallel with the centerline, and a bottom length of 1.2 mm perpendicular to the center line. The maximum height of the protrusions 9 in the channel 6 is 100 μm.

As shown in FIG. 1B and FIG. 1C, the plate-like member 2 and the plate-like member 5 are combined such that the immobilized probe-bearing surface of the probe substrate 1 received and held by the plate-like member 2 via the O ring 4 disposed in the peripheral portion of the hollow 3. The probe substrate 1 comes into contact with the O ring 10 disposed on the bottom surface of the channel of the plate-like member 5. The plate-like members 2 and 5 are fixed together by fixing means (not shown) to form a unit.

Thus, the probe substrate 1 is held, via the O-rings 4 and 10, in the space formed by the plate-like members 2 and 5. As a result, a channel for holding the sample solution, or a channel for circulating the sample solution is formed between the immobilized probe-bearing surface of the probe substrate 1 and the channel 6 of the plate-like member 2 within the unit composed of the plate-like members 2 and 5.

The channel 6 on the immobilized probe-bearing surface of the probe substrate 1 appropriately has a depth of about 20 μm to 250 μm, which is constant all over the channel. The unit composed of the plate-like members 2 and 5 is disposed with an inclination relative to a horizontal plane such that the flow-in port 7 is below the level of the flow-out port 8.

The plate-like member 2 and/or the plate-like member 5 is fitted with a heating/cooling unit 11 for controlling the temperature in the channel. FIG. 1 shows an example in which the plate-like member 2 is equipped with such a heating/cooling unit 11.

Using a feed pump (not shown), the sample solution is caused to flow into the channel through the flow-in port 7 and to flow out thereof through the flow-out port 8 such that the sample solution is circulated through the channel. The flow rate of the circulating sample solution in the channel is, for example, 50 μL/min.

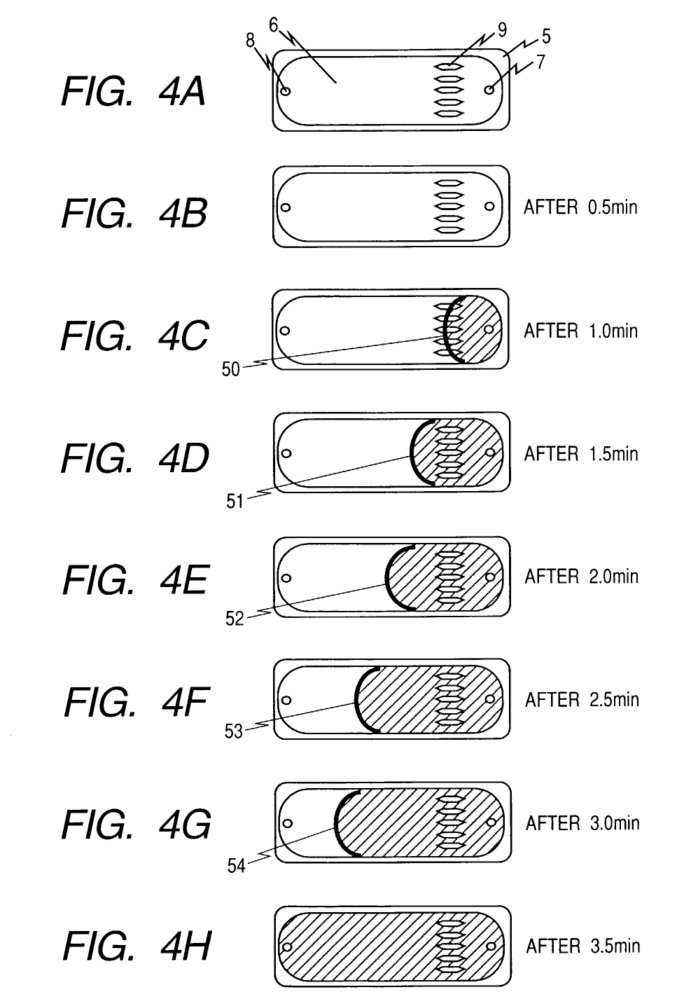

FIGS. 4A to 4H illustrate how the liquid flows in the channel of the circulating type biochemical reaction apparatus of the invention as used in Example 1. A blue dye solution is fed from the flow-in port 7 shown in FIG. 4A at a rate of about 50 μL/min, and the flow of the dye solution is observed at 0.5-minute intervals for 3.5 minutes from the start of feeding of the solution. As shown in FIG. 4C to FIG. 4G, it is confirmed that the front surface (arc-like surface indicated by a black bold line) 50, 51, 52, 53, 54 formed by the flowing dye solution shifted from the side of the flow-in port 7 (lower side of the channel) to the side of the flow-out port 8 (upper side of the channel) almost uniformly and in a parallel manner in proportion to the time of feeding (1.0 to 3.0 minutes). The flow of the blue dye solution is leveled by the disposition of the protrusions 9 as compared with the case of no protrusions therein. It is further confirmed that when the blue dye solution is circulated through the channel, the air previously contained in the channel, bubbles formed during flowing of the blue dye solution into the channel through the flow-in port 7, and bubbles formed during circulation do not remain in the channel but are discharged out of the channel through the flow-out port 8 located at an upper level.

By disposing the flow-in port 7 at a level lower than the flow-out port 8, and by circulating the sample solution in the presence of the protrusions 9, it becomes possible to make the liquid flow in the channel uniformly s as to prevent bubbles, one of the factors making the hybridization reaction inhomogeneous, from accumulating in the channel, and eliminate bubbles from the channel.

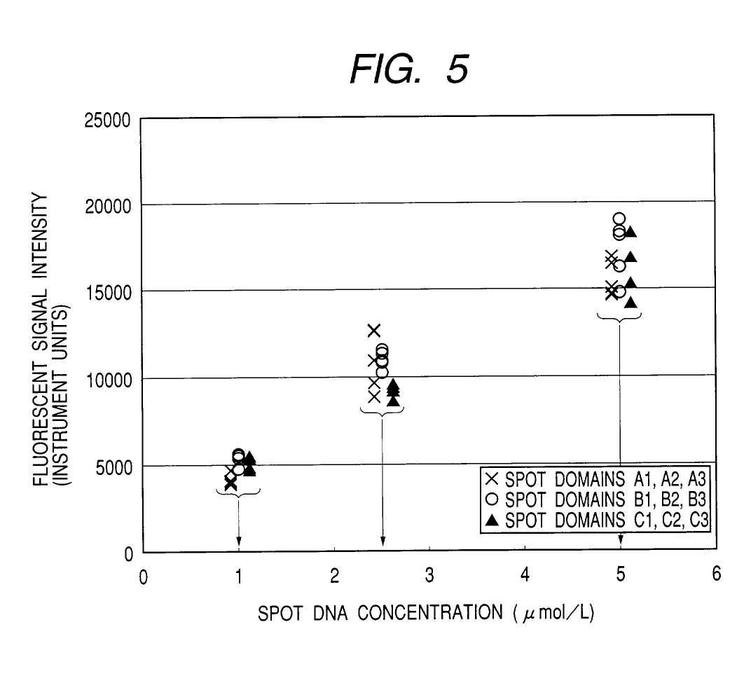

FIG. 5 shows some results obtained by using the circulating type biochemical reaction apparatus of the invention in Example 1, which circulates a sample solution through a DNA microarray prepared by immobilizing, as probe, a DNA on the substrate 1, for effecting hybridization.

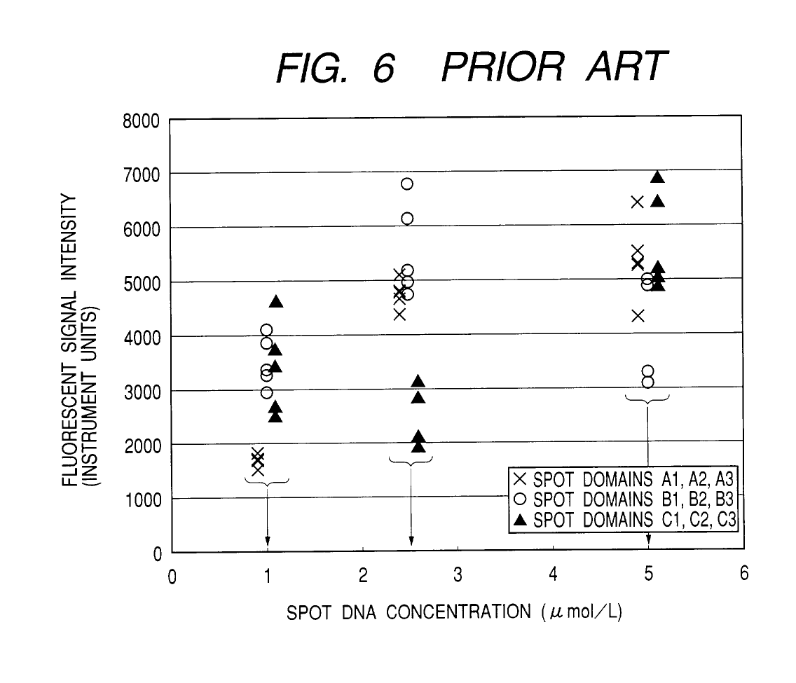

FIG. 6 shows some results obtained in Example 1 by carrying out hybridization with the same DNA microarray as in FIG. 5 but using a reaction apparatus without stirring or circulation.

The DNA microarray is prepared by spotting three solutions, with different concentrations (1, 2.5, and 5 μmol/L), of a synthetic 30-base DNA (probe) having the sequence 1 shown below onto a glass substrate in five sections for each concentration (15 sections in total), followed by immobilization by covalent bonding. The section distribution is shown in FIG. 13. The synthetic DNA is immobilized on the slide glass according to the method of Okamoto et al. as described in Nature Biotechnology, 18 (2000), pp. 438-441. Thus, the slide glass is modified with a maleimide group, and this maleimide group is covalently bonded to the synthetic DNA through crosslinking with the 5′ terminal thiol group of the DNA.

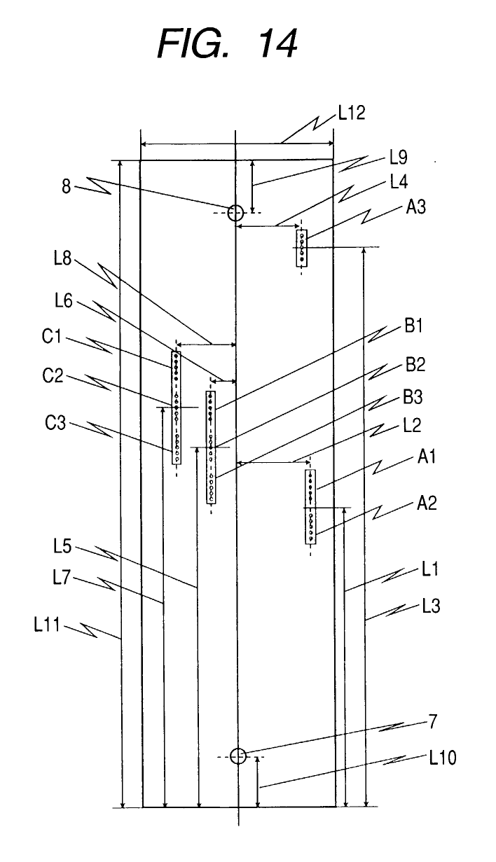

FIG. 14 illustrates the approximate positions on the glass substrate of the domains A1, A2, A3, B1, B2, B3, C1, C2 and C3, including the sections (45 sections in total) on the DNA microarray as measured via fluorescence intensity after the hybridization reaction in Example 1 according to the invention. The domain A1 (probe concentration 5 μmol/L) the domain A2 (2.5 μmol/L) and the domain A3 (1 μmol/L) are located on the glass substrate 1 on the right side of the center line connecting the center of the flow-in port 7 with the center of the flow-out port 8, and L1=350 mm, L2=68 mm, L3=650 mm, and L4=62 mm. The domain B1 (probe concentration 5 μmol/L), the domain B2 (2.5 μmol/L) and the domain B3 (1 μmol/L) are positioned on the left side of the centerline, and L5=420 mm, and L6=25 mm. As for the domain C1 (probe concentration 5 μmol/L), the domain C2 (2.5 μmol/L) and the domain C3 (1 μmol/L), L7=470 mm, and L8=68 mm. The positions of the flow-in port 7 and flow-out port 8 on the substrate corresponded to the positions of L9, L10=66 mm. Further, L11=75 mm, and L12=22 mm.

(Sequence 1)

5° CAAGCTTATCGATACCGTCGACCTCGAGGG 3′

The hybridization against the DNA microarray with the DNA having the sequence 1 immobilized thereon is carried out using a synthetic DNA (target substance) having the sequence 2 (shown below) complementary to the immobilized synthetic DNA and fluorescence-labeled with Texas Red.

(Sequence 2)

5° CCCTCGAGGTCGACGGTATCGATAAGCTTG 3′

In carrying out the hybridization, the concentration of the target substance DNA is adjusted to 0.01 μmol/L, and a sample solution containing the target substance and reagents necessary for hybridization is circulated at a rate of about 50 μL/min at 40° C. for 1 hour.

In a comparative example, the hybridization is carried out at a DNA concentration of 0.01 μmol/L at 40° C. for 6 hours using a reaction apparatus in which the sample solution is neither stirred nor circulated.

After hybridization, for each of the sections (45 sections in total) in the domains A1, A2, A3, B1, B2, B3, C1, C2, and C3 on the DNA microarray as shown in FIG. 14, the fluorescence emitted upon excitation of the fluorescent label is measured. In FIG. 5 and FIG. 6, the ordinate denotes the fluorescence intensity (instrument units), and the abscissa denoted the reference concentration (μmol/L) of the probe solution spotted.

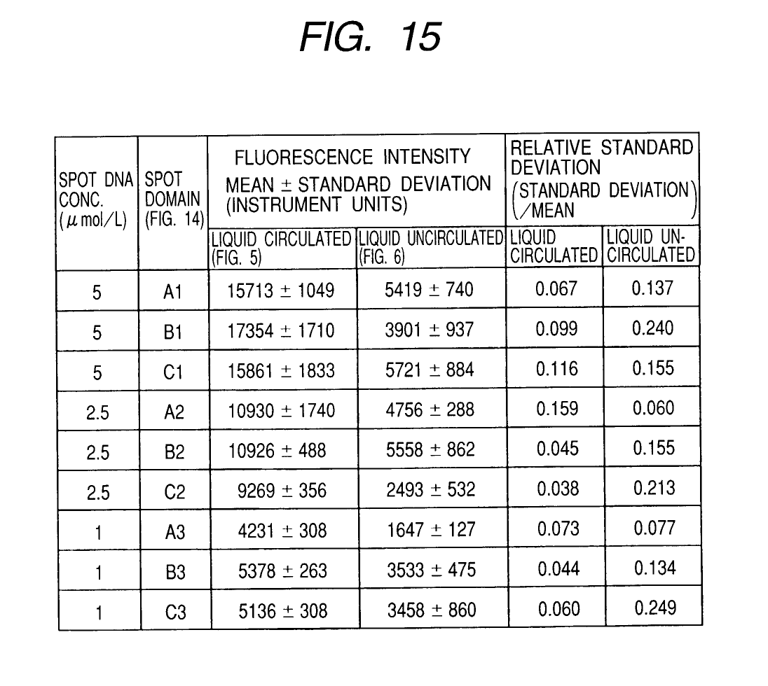

FIG. 15 shows the mean fluorescence intensity values and their standard deviations, and their relative standard derivations as obtained in the respective spot domains in Example 1 according to the invention, with circulation of the sample solution in the step of hybridization (FIG. 5) or with the reaction apparatus in which the sample solution is neither stirred nor circulated (FIG. 6).

The results shown in FIG. 5, FIG. 6, and FIG. 15 revealed the following. When the method comprising a step of circulating the sample solution, in spite of the hybridization time being one sixth of that in the comparative example, (1) the fluorescence intensity detected increases to a level about twice or higher as compared with the comparative example, and (2) the variation in fluorescence intensity is lowered to a level about half or lower as compared with the comparative example. Furthermore, (3) the linearity of the detected fluorescence intensity depending on the spotted DNA concentration is improved. Based on these results, the improvements in hybridization efficiency, reproducibility and quantitativeness as produced by circulating the sample solution are confirmed.

### EXAMPLE 2

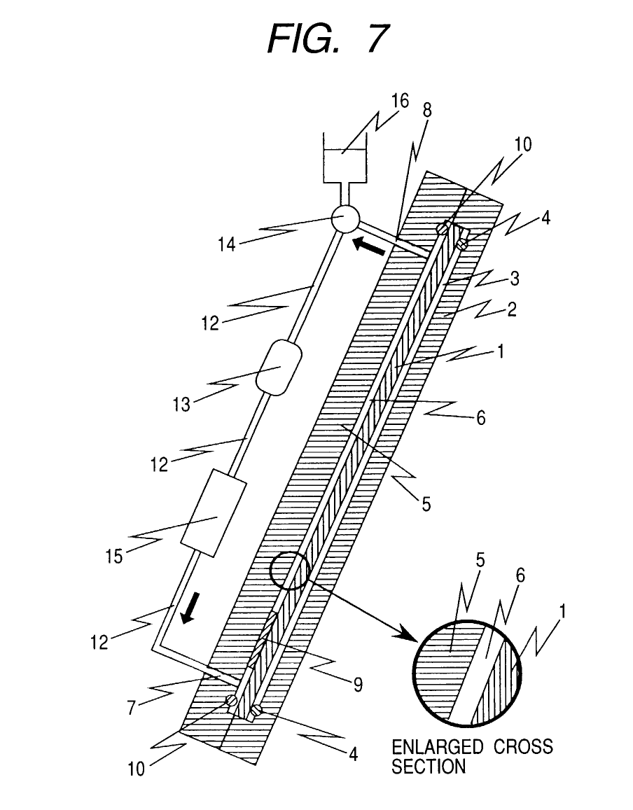

FIG. 7 is a sectional view, at a position corresponding to that of FIG. 1B, showing the constitution of a second embodiment (Example 2) of the circulating type biochemical reaction apparatus of the present invention, which is equipped with a liquid feeding pump. The liquid feeding pump 13 is integrated with the plate-like member 2 or 5 for pouring the sample solution into the apparatus and circulating the sample solution through the channel. The probe substrate 1 is held and disposed with an inclination, relative to a horizontal plane, such that the flow-in port 7 is positioned below the flow-out port 8. A channel 12 is formed so as to connect the flow-in port 7 with the flow-out port 8. The pump 13 for liquid feeding, a channel switch 14, and a bubble-trapping site 15 are disposed within the channel 12.

Since the entrance to the bubble-trapping site 15 is positioned at a level higher than that of the outlet of the site 15, as seen in the direction of gravity, the bubbles, in any, in the sample solution are trapped without being allowed to flow out of the site 15. A sample reservoir 16 for reserving the sample solution is connected with the channel 12 via the channel switch 14. After pouring the sample solution into the sample reservoir 16, the sample reservoir 16 is brought into communication with the channel 12 by operating the channel switch 14.

The sample solution in the sample reservoir 16 is introduced into the channel 12 by the feed pump 13 for sucking up the sample solution, through the flow-in port 7 into the channel formed within the unit composed of the plate-like members 2 and 5. After the channel is formed within the unit composed of the plate-like members 2 and 5, and the channel 12 are filled sufficiently with the sample solution, the fluid communication between the sample reservoir 16 and the channel 12 is cut off by the channel switch 14 to thereby form a closed liquid circulating system formed by the channel within the above-mentioned unit, the sample solution is circulated at a predetermined flow rate for a predetermined period of time. The arrows in the figure indicate the flow direction of the sample solution in the liquid circulation system.

Thus, by using the pump for liquid feeding, the sample reservoir, and the channel switch, it becomes possible to operate the reaction apparatus with our other devices and thus flexibly cope with the change in number of samples to be tested.

### EXAMPLE 3

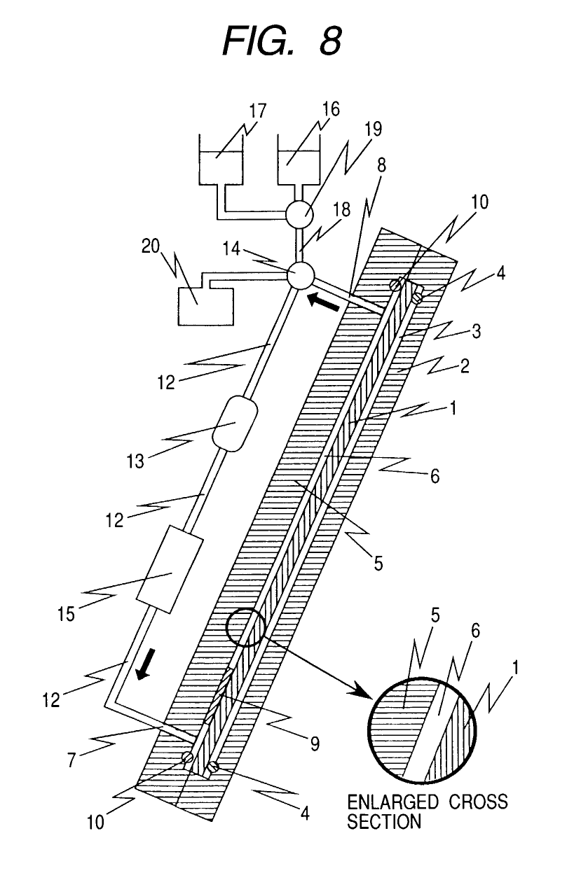

FIG. 8 is a cross-sectional view (at a position corresponding to FIG. 1B) showing the construction of a third embodiment (Example 3) of the circulating type biochemical reaction apparatus of the invention, which is equipped with a washing solution reservoir and a waste liquor reservoir. In addition to the constitution shown in FIG. 7, there are disposed a washing solution reservoir for storing a washing solution for washing the probe substrate and the channel after the reaction of the probe and the target substance, and a waste liquor reservoir for storing the waste liquor after the washing. FIG. 8 shows, as an example, the case where one washing solution reservoir 17 and one waste liquor reservoir 20 are disposed. The number of washing solution reservoirs and that of waste liquor reservoirs each can be arbitrarily selected.

The operation of the apparatus is explained referring to FIG. 8. After completion of the reaction, the washing solution reservoir 17 is brought into communication with a channel 18 by means of a channel switch 19. Then, the channel 18 is brought into communication with the channel 12 by means of the channel switch 14. On this occasion, the waste liquor reservoir 20 is brought into communication with the flow-out port 8 by the channel switch 14. The washing solution is poured into the washing solution reservoir 17.

The liquid feeding pump 13 causes the washing solution to flow from the washing solution reservoir 17 through the flow-in port 7 into the channel 6 formed within the unit composed of the plate-like members 2 and 5, whereby the washing after completion of the reaction between the probe and the target substance is performed. The sample solution after completion of the reaction and the waste liquor are introduced into the waste liquor reservoir 20 and stored there. Since the washing solution can be allowed to flow into the apparatus directly following the completion of the reaction between the probe and the target substance, the irregularities in washing procedure as caused by manual washing operations is eliminated so as to obtain more reliable hybridization results.

### EXAMPLE 4

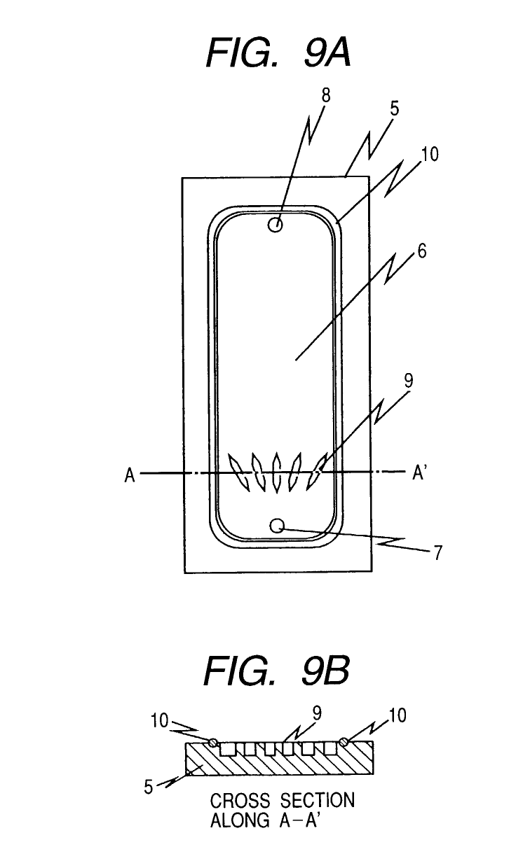

FIG. 9 illustrates another constitution of the protrusions 9 to be formed on the plate-like member 5 (FIG. 3), namely, the constitution thereof in the circulating type biochemical reaction apparatus in Example 4 according to the present invention. FIG. 9A is a plan view of the disposition of hexagonal protrusions 9 formed on the second plate-like member, and FIG. 9B is a cross-sectional view along A-A′ in FIG. 9A.

In FIGS. 9A and 9B, the protrusions are disposed radically in the vicinity of the flow-in port 7. The protrusions 9 are disposed symmetrically relative to the centerline connecting the center of the flow-in port 7 and that of the flow-out port 8.

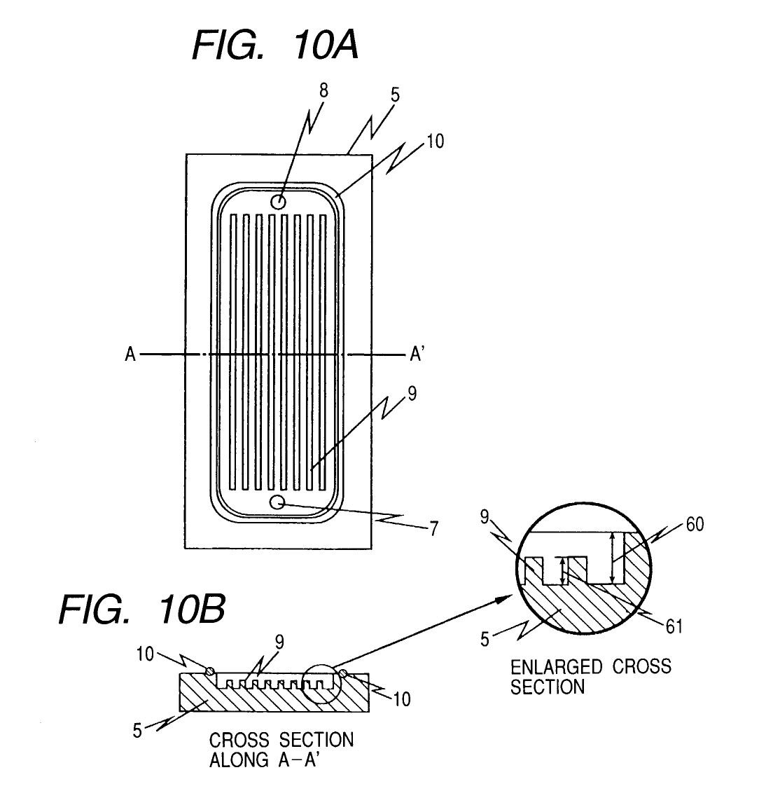

FIGS. 10A and 10B illustrate another example of the constitution of protrusions 9 in lieu of the hexagonal protrusions 9 shown in FIG. 3. FIG. 10A is a plan view showing a plurality of parallel linear protrusions formed all over the channel, and FIG. 10B is a cross-sectional view along A-A′ in FIG. 10A, with a partial enlarged view of the protrusions. The plurality of linear protrusions are disposed symmetrically relative to the centerline. The width of each linear protrusion 9 in parallel with the centerline is about 60 mm. The width of each linear protrusion 9 (perpendicular to the centerline) is about 2.0 mm. The maximum height 61, from the bottom surface, of the linear protrusions 9 is selected such that it is smaller than the depth 60 from the bottom surface to the substrate surface but is greater than one half of the depth 60.

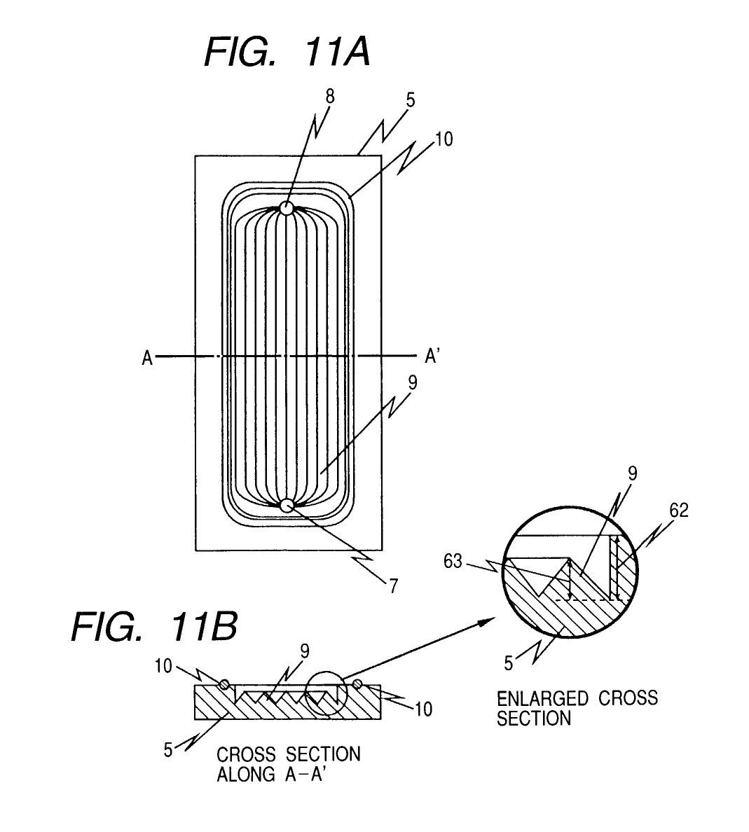

FIGS. 11A and 11B show another example of the constitution of protrusions 9, wherein the linear protrusions 9 shown in FIGS. 10A and 10B are each connected with the liquid flow-in port 7 and with the flow-out port 8. FIG. 11A is a plan view, and FIG. 11B a cross-sectional view along A-A′ in FIG. 11A, with a partial enlarged view of the protrusions. The maximum height 63 of the protrusions 9 from the bottom surface is smaller than the depth 62 from the bottom surface to the substrate but greater than one half of that depth. Referring to FIGS. 10A and 10B and FIGS. 11A and 11B, the shape and height of the protrusions 9 to be disposed and their positions can be selected arbitrarily.

The positions of the probe to be immobilized on the substrate vary according to the number of spots for immobilization and/or the probe species. In particular, when the number of spots is great, the spotted areas account for the majority of the substrate surface such are that the regions where protrusions 9 are to be disposed are restricted. The radial disposition of the protrusions as shown in FIG. 9 can reduce the area of disposition and thus make it possible to dispose probe sections within the limited area of the substrate. Further, as shown in FIGS. 10A and 10B and FIGS. 11A and 11B, by selecting the height of the protrusions such that the upper surface or the upper part of each protrusion may not contact with the substrate surface, it becomes possible to form the protrusions for rectifying the liquid flow all over the channel, and it becomes possible to dispose protrusions to rectify the flow of the solution, irrespective of the non-spotted region (region free of the sections 30).

### EXAMPLE 5

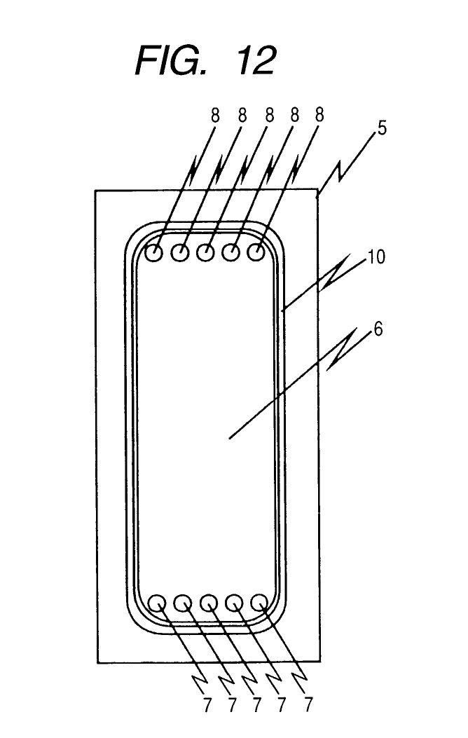

FIG. 12 is a plan view showing an example of the disposition of a plurality of flow-in ports and of flow-out ports as formed on the second plate-like member in the circulating type biochemical reaction apparatus employed in Example 5 according to the invention. While FIGS. 1A to 1C or FIGS. 3A to 3D show an example of the constitution in which one flow-in port 7 and one flow-out port 8 are formed on the plate-like member 5, the constitution shown in FIG. 12 includes four flow-in ports 7 and four flow-out ports 8 formed on the plate-like member 5. The center-to-center lines connecting the flow-in ports 7 with the respective opposed flow-out ports 8 are parallel with each other. While, in FIG. 12, only four flow-in ports and four flow-out ports are formed in parallel, the number of flow-in ports and that of flow-out ports may be selected arbitrarily. By disposing a plurality of flow-in ports and of flow-out ports, it becomes possible to make the flow within the reaction channel uniform so as to improve the hybridization efficiency and reduce the variation in signal intensity, regardless of the presence or absence of protrusions.

By using the circulating type biochemical reaction apparatus according to the invention for circulating the sample solution containing biomolecules interacting with a probe immobilized on the substrate through the channel, the reaction efficiency can be improved so as to increase the signal intensity and reduce the reaction time. Further, as a result of uniform circulation of the sample solution and the removal of bubbles from the sample solution, it becomes possible to allow the reaction to proceed uniformly and reduce the variation in signal intensity. In cases where a plurality of circulating type biochemical reaction apparatus according to the invention are used, it becomes possible to carry out experiments while flexibly coping with the change in the number of samples to be tested, when each circulating type biochemical reaction apparatus is provided with a liquid feeding pump and a temperature control unit.

What is claimed is:

1. A circulating type biochemical reaction apparatus comprising:
a first plate-like member for holding a substrate which has at least one probe immobilized thereon for selectively binding therein a target substance in a sample solution;
a second plate-like member having at least one flow-in port for guiding the sample solution containing the target substance to flow into a respective internal channel and at least one flow-out port for guiding the sample solution containing the, target substance to flow out the internal channel; and
at least one respective external channel connected with the internal channel via the flow-in port and the flow-out port to form a loop for circulating the sample solution,
wherein the internal channel is formed between an immobilized probe-bearing surface of the substrate and the second plate-like member,
wherein the second plate-like member is constructed such that the flow-in port is disposed below the flow-out port.

2. A circulating type biochemical reaction apparatus as claimed in claim 1, wherein the first plate-like member and the second plate-like member are disposed with an inclination from a horizontal plane.

3. A circulating type biochemical reaction apparatus as claimed in claim 1, further comprising a pump for circulating the sample solution, said pump is installed integrally with the loop.

4. A circulating type biochemical reaction apparatus as claimed in claim 1 which further comprises temperature controlling means for heating and/or cooling the loop.

5. A circulating type biochemical reaction apparatus as claimed in claim 1, further comprising a site for trapping bubbles in the sample solution flowing in the loop.

6. A circulating type biochemical reaction apparatus as claimed in claim 5, wherein the bubbles are moved upward by disposed the substrate, the first plate-like member and the second plate-like member at a predetermined angle from a horizontal plane so as to be carried along the loop to the site for trapping bubbles.

7. A circulating type biochemical reaction apparatus as claimed in claim 6, wherein an entrance to the bubble trapping site is disposed higher than an outlet of the bubble trapping site.

8. A circulating type biochemical reaction apparatus as claimed in claim 1, further comprising at least one channel switch means for selectively connecting a reservoir to the loop.

9. A circulating type biochemical reaction apparatus as claimed in claim 1, further comprising at least one reservoir connected to the loop.

10. A circulating type biochemical reaction apparatus as claimed in claim 1, further comprising protrusions for controlling a flow of the sample solution in the internal channel, said protrusions are formed on a surface of the second plate-like member which comes into contact with the sample solution.

11. A circulating type biochemical reaction apparatus as claimed in claim 10, wherein the protrusions are formed in the vicinity of the flow-in port.

12. A circulating type biochemical reaction apparatus as claimed in claim 10, wherein a plurality of linear protrusions are formed on that surface of the second plate-like member.

13. A circulating type biochemical reaction apparatus as claimed in claim 10, wherein a plurality of linear protrusions are formed on that surface of the second plate-like member, while one end of each linear protrusion inclines to the flow-in port and the other end of each linear protrusion inclines to the flow-out port.

14. A circulating type biochemical reaction apparatus as claimed in claim 12, wherein a maximum height of the linear protrusions is smaller than a depth from the surface of the second plate-like member to the substrate surface but is greater than one half of the depth.

15. A circulating type biochemical reaction apparatus as claimed in claim 13, wherein a maximum height of the linear protrusions is smaller than a depth from the surface of the second plate-like member to the substrate surface but is greater than one half of the depth.

16. A circulating type biochemical reaction apparatus as claimed in claim 1, wherein a distance between a surface of the second plate-like member which comes into contact with the sample solution and the immobilized probe-bearing surface of the substrate is constant.

17. A circulating type biochemical reaction apparatus as claimed in claim 16, wherein said distance is 20 μm to 250 μm.

18. A circulating type biochemical reaction apparatus as claimed in claim 1, wherein the target substance is a single-stranded or double-stranded nucleic acid, an antibody, an antigen, a receptor, a ligand, or an enzyme, whereas the probe is a nucleic acid or peptidyl nucleic acid, an antigen, an antibody, a ligand, a receptor, or a substrate, respectively.

19. A method for circulating a sample solution of a biochemical reaction comprising a step of pumping the sample solution against gravity along a loop within a circulating type biochemical reaction apparatus thereby circulating the sample solution in the loop without accumulating bobbles therein.

20. A method for circulating a sample solution of a biochemical reaction as claimed in claim 19, further comprising a step of inclining a longer side of the loop from a horizontal plane.
